(19) United States
(12) Patent Application Publication
(10) Pub. No.: US 2025/0342423 A1
(43) Pub. Date: Nov. 6, 2025

(54) DRUG PRODUCTION METHOD

(71) Applicants: Hoffmann-La Roche Inc., Little Falls, NJ (US); Genentech, Inc., South San Francisco, CA (US)

(72) Inventors: Christian Song-Hyo SCHMID, Basel (CH); Paolo SOUMPALAS, Zug (CH); Yang TANG, Mississauga (CA); Antti Ilmari AHONEN, Sarnen (CH); Yiming PENG, SouthSanFrancisco, CA (US)

(21) Appl. No.: 18/859,952

(22) PCT Filed: Apr. 25, 2023
(86) PCT No.: PCT/EP2023/060778

§ 371 Date: Oct. 24, 2024

(30) Foreign Application Priority Data
Apr. 25, 2022 (EP) 22169845.9

(51) Int. Cl. G06Q 10/0631; G06Q 50/04

(52) U.S. Cl. CPC G06Q 10/06316; G06Q 50/04

(57) ABSTRACT

A method for producing at least one drug product using a lyophilization step. The method comprises receiving, at a computing system, process requirements of a drug production process for the at least one drug product to be produced. The drug production process comprising at least one pre-processing step, the lyophilization step, and at least one post-processing step. The process requirements include the required steps of the drug production process for the/each drug product, operator requirements of each step, and an approximate duration of each step. The method further comprises receiving, at the computing system, downtime constraints of the equipment required for the drug production process, wherein the downtime constraints comprise time periods when the lyophilization step cannot be initiated, but where at least one or more steps, or portions of steps, of the drug production process can be implemented; identifying, at the computing system, a total duration of the drug production process for a plurality of different candidates for a sequence of steps of the drug production process, based on the process requirements and the downtime constraints; identifying, at the computing system, a shortest sequence of steps of the drug production process by selecting, from the plurality of different candidates, the sequence of steps with the shortest total duration as the shortest sequence of steps; and implementing the drug production process according to the shortest sequence of steps in order to produce the at least one drug product.

### CROSS-REFERENCE TO RELATED APPLICATIONS

This application is a U.S. National Stage application under 35 U.S.C. § 371 of International Application No. PCT/EP2023/060778, filed internationally on Apr. 25, 2023, which claims priority to and benefit of European Patent Application No. 22169845.9, filed on Apr. 25, 2022, the contents of each of which are hereby incorporated herein by reference in their entirety and for all purposes.

### FIELD OF THE INVENTION

The present invention relates to a method for producing a drug product and particularly, although not exclusively, to a method for producing a drug product wherein the method comprises a lyophilization step.

### BACKGROUND

Producing drug products comes with many challenges. In particular, a drug production line generally includes multiple steps, each of which may span hours or even days to complete, and which may each require various pieces of different equipment, and differing amounts of manual operation.

Some production lines also produce multiple different drug products. Each different drug product may have a specific format and process duration. For example, the duration of a lyophilization step in the drug production process may differ depending on the drug product being produced.

Lyophilization is a freeze-drying process step, wherein the drug product is frozen, placed in a vacuum, and the water therein removed. Lyophilization is generally used in the production of drug products where the bulk drug ingredients are not stable in frozen or liquid form, e.g. due to degradation, biological growth, heat sensitivity, chemical reactions etc. It can help to enable a longer shelf life of the drug product, easier transportation of the drug product, and can allow for drug products to be stored at room temperature.

However, there are some disadvantages associated with lyophilization. The lyophilization step itself is timely, generally having a duration of between 10 and 100 hours for a single product batch. Furthermore, the lyophilization process may require multiple pre-processing and handling steps, which may include a compounding step and a filling step performed before the lyophilization step, and an unloading step following the lyophilization step. Each of these steps are also timely, with an approximate duration of between 5 and 20 hours each, thus further increasing the duration of the drug production method.

Further still, the lyophilization process can be messy, and may contaminate the equipment and the surrounding area. As such, in production lines handling multiple different drug products, a washdown step is also required between format changes. This results in a downtime of the lyophilization equipment, where no lyophilization can take place. This downtime may be between approximately 10 and 20 hours.

Accordingly, introducing lyophilization into a drug production line can greatly increase the duration of the drug production process.

Further time delays can also occur due to equipment downtime (i.e., when the equipment cannot be used in the drug production process). This may be due to equipment availability. For example, there may be equipment downtime during cleaning of the equipment, as mentioned above, due to equipment maintenance, or due to the equipment being used in a different step already being performed. Furthermore, some steps of the drug production line, or at least the initialization of some steps, may require manual user input. This can only be performed when the operators are available to perform the manual user input. As such, operator availability, and in particular lack of availability, may also cause delays to the drug production process, further increasing the duration of the drug production line.

This increased duration of the drug production process due to the lyophilization step has led some drug production facilities to consider removing the lyophilization step altogether and to instead attempt to find alternative processing steps to enable a stable final drug product. However, these alternative processing steps often have further disadvantages. The result achieved by the lyophilization process is often preferred over these alternative processing steps.

The present invention has been devised in light of the above considerations.

### SUMMARY OF THE INVENTION

In a first aspect, there is provided a method for producing at least one drug product using a lyophilization step, the method comprising:
receiving, at a computing system, process requirements of a drug production process for the at least one drug product to be produced, the drug production process comprising at least one pre-processing step, the lyophilization step, and at least one post-processing step, wherein the process requirements comprise:
   the required steps of the drug production process for the/each drug product;
   operator requirements of each step; and
   an approximate duration of each step;
receiving, at the computing system, downtime constraints of the equipment required for the drug production process, wherein the downtime constraints comprise time periods when the lyophilization step cannot be initiated, but where at least one or more steps, or portions of steps, of the drug production process can be implemented;
identifying, at the computing system, a total duration of the drug production process for a plurality of different candidates for a sequence of steps of the drug production process, based on the process requirements and the downtime constraints;
identifying, at the computing system, a shortest sequence of steps of the drug production process by selecting, from the plurality of different candidates, the sequence of steps with the shortest total duration as the shortest sequence of steps; and
implementing the drug production process according to the shortest sequence of steps in order to produce the at least one drug product.

In this way, the batch capacity of the at least one drug product can be increased by optimizing the production plan and asset utilization of the lyophilization step without modifying the constraints and process durations, by better utilizing the periods of time where the lyophilization process cannot be initiated, but where other (portions of) steps can be implemented. It has been found that the batch capacity of a lyophilization production line, from compounding the drug substance through to unloading filled vials of the drug product from a lyophilizer, can be increased by up to 20% by optimizing the production process without changing the process requirements or the downtime constraints. This is possible because the method identifies and then implements the quickest, or optimal, sequence of steps that minimizes downtimes of the equipment and therefore increases asset utilization and production capacity. Ultimately, this enables more drug products to be produced, increasing the production capacity, whilst still obtaining the preferred quality resulting from inclusion of the lyophilization step.

Optional features will now be set out. These are applicable singly or in any combination with any aspect of the invention.

As used herein, a lyophilization step may comprise a freeze-drying process step, wherein a drug product is frozen, placed in a vacuum, and the water therein removed.

The drug product may be a biological drug product, such as an anti-body drug conjugate, for example.

The at least one pre-processing step may be implemented before the lyophilization step. The at least one pre-processing step may comprise a compounding step. The compounding step may include combining, mixing or altering ingredients or constituents of the drug product. The at least one pre-processing step may alternatively/additionally include a filling step, which may involve filling the compounded drug substance into containers, such as vials, for the lyophilization step. Further pre-processing steps which may be included comprise one or more of a compounding preparation step, a filling preparation step, and a lyophilization preparation step.

The at least one post-processing step may be implemented after the lyophilization step. The at least one post-processing step may comprise an unloading step, in which the at least one drug product may be unloaded, e.g., from vials, following the lyophilization step.

Alternatively/additionally, the at least one post-processing step may comprise a cleaning (e.g. washdown) step, e.g., to clean the containers/vials following lyophilization.

Accordingly, the process requirements of the drug production process for the/each drug, which are received at the computing system, may include which of the pre-processing and post-processing steps are required along with the lyophilization step, for the/each drug product.

The process requirements may also include a predefined order of the required steps of the drug production process for the/each drug product.

The process requirements may also include a predefined format, and/or size of the batch, of the/each drug product to be produced.

The operator requirements of each step, which are received at the computing system, may include information indicative of the operator input required for the step (or portion thereof), and, optionally, at what stage/for what duration of the step. For example, manual operator input may be required at the initiation of the lyophilization step, but then may not be needed for the remainder of the duration of the lyophilization step (such that the lyophilization step can continue even if an operator is not present or available).

The approximate duration of each step may be based on historical data indicative of the actual duration of each step when implemented previously in the production of the same drug product, e.g., an average of the actual durations of the step when implemented previously. An average (e.g. mean, median) of previous actual durations of the step may be used, because the duration of the step may vary based on external factors, such that the duration of the step may not always be consistent.

Thus, the method may comprise, for each step, receiving historical data indicative of the actual duration of the respective step when implemented previously, determining an average of the historical data, and determining the approximate duration of the respective step based on the average (e.g. such that the approximate duration is equivalent to the average).

In this way, the historical data used to calculate the average duration of each step can be updated over time as new data becomes available (e.g., as the steps are repeatedly implemented). The determined average duration of each step may therefore become a more accurate representation of the step durations, e.g., as the operators adapt to performing the steps (or portions of the steps) over time, or as other external (e.g., environmental) conditions change over time.

Optionally, the historical data may be limited to historical data of the actual duration of the steps implemented under the same downtime constraints as those received at the computing system. Advantageously, this allows for the simulation to better map the relationship between the durations of the steps of the drug production process for that specific drug product and the downtime constraints. This allows for more accurate values for the approximate durations of each step, and thus a more accurate identification of the shortest sequence of steps of the drug production process, and hence a higher batch capacity.

Alternatively, the approximate duration of each step received at the computing system may comprise predefined fixed values.

The downtime constraints may comprise information indicative of equipment availability. For example, downtime constraints may include time periods when the equipment is unavailable, such as equipment maintenance periods and/or equipment cleaning periods, which may prevent pieces of equipment required for one or more of the steps (or portions of a step) to be used. The downtime constraints may alternatively/additionally include information indicating that the equipment is already in use in a different step.

The time periods when the lyophilization step cannot be initiated, but where at least one or more steps, or portions of steps, can be implemented, may be based on operator availability. For example, the operator availability may include information on shift patterns or working times of the operator(s). This allows for time periods where an initialization of the lyophilization step cannot be initiated (e.g., due to operator unavailability) to be used for other steps of the drug production process.

The process requirements and downtime constraints may be received at the computing system via a user interface (e.g. a keyboard, touch screen, microphone, etc.) and/or via a wired or unwired connection. The process requirements may be received from a storage medium, which may be at the computing system (e.g., may form part of the computing system itself) or may be remote from the computing system (e.g., from a remote server).

The different candidates may comprise the sequence of steps in the drug production process being performed at different times, and/or in different orders for example.

Identifying the shortest sequence of steps may comprise identifying a start time which provides the implementation/performance of the drug production process with the shortest total duration (e.g. compared to the remaining different candidates). As such, the constraints of the operator requirements and the downtime constraints may be used (e.g. optimized) to minimize unwanted delays when the drug production process is implemented.

Optionally, the process requirements may also comprise a predefined start time and/or a predefined end time of the entire drug production process. The process requirements may comprise a predefined start time and/or a predefined end time of one or more steps of the drug production process.

Identifying the shortest sequence of steps of the drug production process may comprise identifying a maximum batch capacity of the drug product based on the process requirements and the downtime constraints (including the predefined start and end time). As such, based on the predefined start and end time of the drug production process, the sequence of steps may be optimized based on the constraints to minimize downtime of the equipment.

The method may be for producing at least two different drug products, the required steps of the drug production process for each drug product comprising a lyophilization step. This may improve the efficiency of the drug production process by using the same equipment for producing both drug products.

The process requirements may include the required steps of the drug production process for each of the at least two drug products. The different drug products may have different formats. The steps in the drug production process for the different drug products may have different approximate durations.

The process requirements may include sequence requirements, for example a requirement that one of the drug products must be produced within a predefined time period.

Identifying a shortest sequence of the steps of the drug production process for the at least two drug products may comprise identifying an order for producing the at least two drug products which results in the shortest sequence of steps, based on the process requirements and the downtime constraints (e.g., the quickest order).

Identifying the order for producing the at least two drug products which results in the shortest sequence of steps may comprise identifying the total duration of the sequence of steps of the drug production process for the at least two drug products for a plurality of different candidates of the order of steps, and selecting the sequence of steps with the shortest total duration as the (quickest) order.

It has been found that selecting the optimum ordering of the production of the two drug products based on the process requirements and the downtime constraints can reduce the total duration substantially, without modifying the constraints or process requirements. This may allow for increased batch production. For example, for drug product A and drug product B, the methods described herein may identify that it is quicker to produce drug product A then drug product B, than to produce drug product B then drug product A.

The method may comprise outputting the identified shortest sequence of steps of the drug production process, e.g., for display. The identified shortest sequence of steps of the drug production process may be displayed, e.g., on a display screen. It may be displayed as a Gantt chart, for example.

The method may further comprise, before implementing the drug production process according to the identified shortest sequence of steps, identifying, at the computing system, a predicted total duration of the drug production process based on the identified shortest sequence of steps and historical data indicative of an actual duration of each step (or the sequence of steps) when implemented previously in the production of the same drug product(s).

The method may comprise outputting the predicted total duration of the drug production process, e.g., for display.

The method may comprise outputting (e.g., for display) the sequence of steps of the drug production process for a plurality (e.g., some or all) of the different candidates. They may be displayed as Gantt charts, for example. The total duration of the plurality of different candidates may also be displayed, e.g., for comparison by a user. This may allow the user to select a sequence of steps of the drug production process (e.g., the shortest sequence of steps), as the sequence of steps by which to implement the drug production process.

Other additional information related to each candidate sequence of steps may also be displayed. For example, the method may also comprise outputting for display, for a plurality of different candidates (e.g., a predefined number of the plurality of candidates having the shortest sequence, such as the 5 quickest candidates), information related to the number of times user input is required during the sequence of steps, an estimated cost of implementing the sequence of steps, and/or a date and/or time at which the sequence of steps is scheduled to start and/or end. The user can then use this additional information in a decision of which candidate of the sequence of steps to implement.

The method may comprise comparing the predicted total duration of the drug production process to a predefined duration threshold, and implementing the drug production process according to the identified shortest sequence of steps only when the predicted total duration of the drug production process is less than the predefined duration threshold. In this way, the drug production process may only be implemented when the predicted total duration time is less than a predefined limit (e.g., when it is predicted that the process will finish within an operator's availability). This may reduce the need for operators to extend their working hours, for example. This comparison may be performed by the computing system, for example.

Optionally, the predicted total duration of the drug production process may be identified (e.g., by the computing system) a plurality of times, using different historical data indicative of an actual duration of each step when performed previously in the production of the same drug product(s). The actual durations of a respective step implemented previously (e.g. in multiple drug production processes for the same drug product) may differ, e.g., based on external factors such as efficiency of the operators, or environmental conditions. Thus, when the predicted total duration is identified multiple times, the predicted total duration may differ based on this differing historical data.

The method may comprise determining, at the computing system, an average (e.g. mean, median, 25% or 75% quantile) of the plurality of predicted total times and comparing the average to a predefined duration threshold, as detailed above.

Alternatively, the method may comprise comparing each of the plurality of predicted total times to a predefined duration threshold, and identifying an indicator representing the percentage of times the predicted total time is less than (or more than) the predefined duration threshold. This indicator may indicate the probability of implementing the drug production process according to the identified shortest sequence of steps within a predefined time duration. This may be useful in indicating the probability of the process overrunning or finishing within a preferred timeframe. This may reduce the need for operators to extend their working hours, for example. This comparison may be performed by the computing system, for example.

The method may comprise outputting the indicator, e.g., for display.

Optionally, the historical data used in the predicted total duration of the drug production process may be limited to historical data of the duration of the steps implemented under the same downtime constraints as those received at the computing system. Advantageously, this allows for the simulation to better map the relationship between the durations of the steps of the drug production process for that specific drug product and the downtime constraints. This allows for more accurate values for the approximate durations of each step, and thus a more accurate determination of the total duration of the identified shortest sequence of steps.

The method may comprise implementing the drug production process according to the identified shortest sequence of steps only when the indicator is greater than a predefined indicator threshold. In this way, the drug production process may only be implemented when there is a high chance of the drug production process being implemented within a predefined time period. This may reduce the need for operators to extend their working hours, for example.

In some examples, the method may comprise, before implementing the drug production process according to the identified shortest sequence of steps, identifying, at the computing system, a predicted total duration of the drug production process based on the identified shortest sequence of steps and predefined fixed values for the duration of each step.

A further advantage of the above described method is that unplanned events can be accounted for. In particular, the above described method allows for a portion of the drug production process to be optimized, e.g., if the drug production process has been unexpectantly stopped mid-way through implementation due to unplanned events. As such, the production line can be resumed more quickly following unplanned events.

According to a second aspect, there is provided a system configured to perform the method of the first aspect. The system may comprise a computing system comprising one or more processors, the one or more processors configured to perform the method of the first aspect. The computing system may comprise one or more computing devices which may be located at one or more respective locations. For example, the computing system may comprise only one computing device, or may comprise a network of interconnected computing devices (e.g., servers, processors, mobile devices, etc.). The computing system may comprise one or more processors and one or more memory devices configured to store instructions for performing the method of the first aspect.

The one or more memory devices may store the process requirement and downtime constraints for use in the identification of the shortest sequence of steps.

The computing system may be configured to automatically perform the method of the first aspect (e.g., without operator input). Alternatively, user input may be required for the computing system to perform some of the method steps.

Further aspects of the invention include a non-transitory computer-readable storage medium containing machine executable instructions which, when executed on a processor, cause the processor to perform the method of the first aspect.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 is a flow chart illustrating steps in a drug production process including a lyophilization step;

FIG. 2 is a flow chart of a method for producing at least one drug product using a lyophilization step;

FIG. 3 is a schematic of a system for performing the method of FIG. 2;

FIG. 4 is a Gantt chart showing a plurality of different sequences of steps of the drug production process of FIG. 1;

FIG. 5 is a bar chart showing a plurality of different sequences of steps of a drug production process for producing 2 drug products using a lyophilization step;

FIG. 6A is a Gantt chart showing a sequence of steps of a drug production process when the method of FIG. 2 is not performed;

FIG. 6B is a Gantt chart showing an example shortest sequence of steps of a drug production process when the method of FIG. 2 is performed;

FIG. 6C includes a bar chart and Gantt chart showing another example shortest sequence of steps of a drug production process when the method of FIG. 2 is performed; and

FIG. 6D includes a bar chart and Gantt chart showing another example shortest sequence of steps of a drug production process when the method of FIG. 2 is performed.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Lyophilization is a freeze-drying process step used in drug production lines. It comprises freezing the drug substance, placing the drug substance in a vacuum and removing the water therefrom. It is generally used in the production of drug products where the bulk drug ingredients are not stable in frozen or liquid form, e.g. due to degradation, biological growth, heat sensitivity, chemical reactions etc. It can help to enable a longer shelf life of the drug product, easier transportation of the drug product, and can allow for drug products to be stored at room temperature.

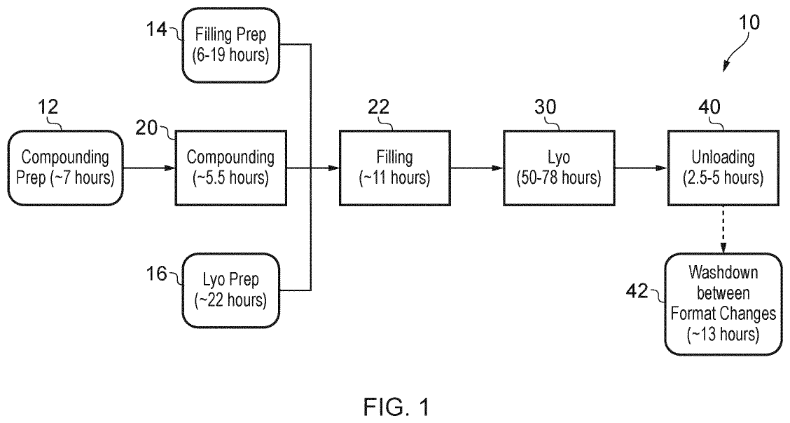

An example drug production line 10, such as that shown in FIG. 1, includes a lyophilization step 30. The lyophilization process may also require a number of pre-processing steps performed before the lyophilization step 30. The pre-processing steps may include a compounding step 20, which may include combining mixing or altering the ingredients or constituents of the drug product, and a filling step 22, which may include filling the compounded drug substance into containers, such as vials, for the lyophilization step 30.

The drug production line 10 also includes further pre-processing steps, including a compounding preparation step 12, a filling preparation step 14, and a lyophilization preparation step 16. Each of these pre-processing steps may be performed in sequence (e.g., one after the other), or two or more of the pre-processing steps may be performed at the same time. For example, the filling step 22 may be performed after the compounding step 20, but the lyophilization preparation step 16 may be performed alongside the compounding step 20 and the filling step 22.

The lyophilization process may also require a number of post-processing steps performed after the lyophilization step 30, the post-processing steps including an unloading step 40, in which the drug product may be unloaded, e.g., from vials, from the lyophilizer (the equipment that used in the lyophilization step 30). The drug production line 10 may also include further post-processing steps, for example, a washdown step 42. In the washdown step 42, the equipment used in the lyophilization step 30 may be cleaned. This is of particular importance when different drug products, e.g. with different formats, are being produced by the drug production line.

Drug production line 10 may be used to product one or more biological drug products, such as anti-body conjugates, for example.

Each of the steps of the drug production line 10 have a respective duration, which may depend on the batch size and/or the drug product format being produced. For example, the compounding step 20 may have a duration of between approximately 4 and 7 hours, or approximately 5.5 hours. The filling step 22 may have a duration of between 10 and 12 hours, or approximately 11 hours. The lyophilization step 30 may have a duration of between approximately 10 and 100 hours, or between approximately 40 and 90 hours, or between approximately 50 and 78 hours. The unloading step 40 may have a duration of between approximately 1 and 8 hours, or between approximately 2.5 and 5 hours. The compounding preparation step 12 may have an approximate duration of between approximately 6 and 8 hours, or approximately 7 hours. The filling preparation step 14 may have a duration of between approximately 4 and 25 hours, or approximately 6 and 19 hours. The lyophilization preparation step 16 may have an approximate duration of between 15 and 30 hours, or approximately 22 hours. The washdown step 42 may have an approximate duration of between 10 and 15 hours, or approximately 13 hours.

Thus, the total duration of the drug production line 10 may extend over many days. This means that the production of the drug product may extend over periods of time where operators an unavailable (e.g. overnight, weekends), or where equipment is unavailable (e.g., due to cleaning or maintenance).

In particular, the extended length of the lyophilization step 30 can be problematic, because operators are often required to initialize the lyophilization step 30. Thus, if the pre-processing steps are completed when the operators or equipment are unavailable, the drug production line 10 is halted, until the operators/equipment are available again. This can cause substantial delays to the drug production process, and thus a reduction in batch capacity.

However, once started, the lyophilization step 30 can be performed without operator input, and thus the extended duration of the step (between approximately 50 and 78 hours) can run whilst the operators are unavailable (e.g., the lyophilization step 30 can run over the weekend when the operators are unavailable).

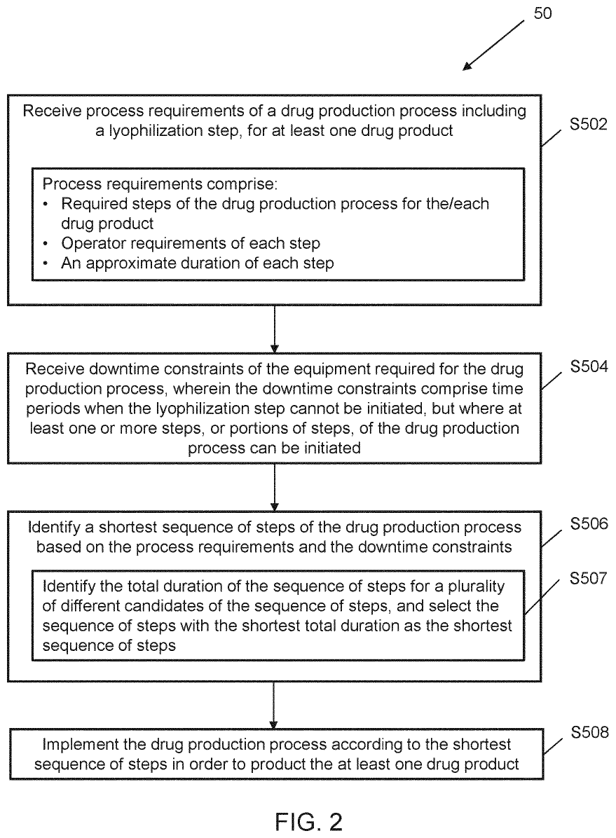

This realization allows the method 50 of FIG. 2 to provide more efficient production of drug products, with improved batch capacity, without altering any operator or equipment constraints.

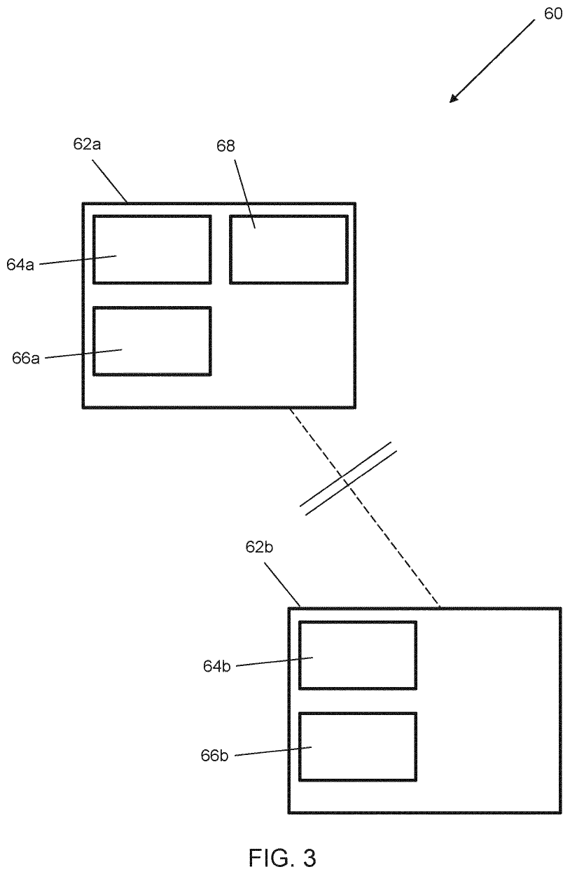

In step S502 of FIG. 2, process requirement of a drug production process including the lyophilization step 30 are received at a computing system 60 (such as that shown in FIG. 3). The drug production process may be for one or a plurality of drug products. The/each drug product may be produced via the drug production line 10 described above. Thus, where multiple drug products are produced, the drug production process may include multiple drug production lines 10 (and thus multiple lyophilization steps 30). The step durations and/or the steps required may differ for different drug products.

The process requirements comprise the required steps of the drug production process for the or each drug product. For example, the computer system 60 may receive information indicative of which of the pre-processing and post-processing steps are required in the production line of each of the drug products. The computer system 60 may also receive information indicative of a predefined order of the required steps for the/each drug product. The computer system 60 may further receive information indicative of a predefined format (e.g., drug product type, amount/weight of ingredients in the drug product) and/or batch size of the/each drug product to be produced.

The process requirements received at S502 also comprise operator requirements of each step of the drug production process. The operator requirements may include information indicative of the operator input required for each respective step, and/or a portion of each step. For example, the operator requirements may indicate that an operator is required for the initialization of the lyophilization step, but that no operator is required thereafter.

The process requirements received at S502 also comprise an approximate duration of each step. This will be described in further detail below.

The process requirement may also comprise a predefined start time and/or a predefined end time of the drug production process.

At S504 of method 50, downtime constraints of the equipment required for the drug production process are received at the computing system 60. The downtime constraints comprise time periods when the lyophilization step 30 cannot be initiated, but where at least one or more steps, or portions of steps of the drug production process can be initiated.

For example, the time periods may correspond to time periods where the operators are unavailable, for example over evenings and weekends. During these time periods, the lyophilization step 30 cannot be initiated (because operator input is required) but a later portion of the lyophilization step 30 (e.g., after initialization) can be performed.

The downtime constraints may also comprise information indicative of equipment availability, for example time periods when the equipment is unavailable such as equipment maintenance periods and/or cleaning periods. These time periods may prevent the respective pieces of equipment being used for any step.

S502 and S504 of method 50 may be performed in any order, e.g., in sequence, or at the same time.

Returning to S502, the approximate duration of each step may comprise predefined fixed values.

For each step, the approximate duration of each step may be based on historical data indicative of the actual duration of that step when implemented previously, e.g., in the production of the same drug product as that in the current drug production process. The approximate duration may be based on an average (e.g., mean, or median) of this historical data.

Thus, in order to determine the approximate duration of a step, the method may comprise receiving the historical data indicative of the actual duration of the respective step when implemented previously (in the production of the same drug product), determining an average of the historical data, and determining the approximate duration of the respective step based on the average, such that the approximate duration is used as the average.

Optionally, for each step, the historical data may be limited to historical data indicative of the actual duration of the step implemented under the same downtime constraints as those received at S504.

This allows for the identification of the shortest sequence to better map the relationship between the durations of the steps of the drug production process for that specific drug product and the downtime constraints. This allows for more accurate values for the approximate durations of each step, and thus a more accurate identification of the shortest sequence of steps of the drug production process, and hence a higher batch capacity.

By using this historical data to calculate the average duration of each step, the determined average duration of each step may become more accurate over time, as the historical data used is updated (e.g., as the steps are repeated). This may be because more historical data is available, or because the average duration of the respective step changes over time, e.g., due to external (such as environmental) conditions changing over time, or because different operators are performing at least portions of the step.

The computing system 60 where the process requirements and downtime constraints are received may comprise one or more, or two or more (e.g., a plurality) of computing devices 62a, 62b. The example shown in FIG. 3 has two computing devices 62a, 62b which may be positioned at remote locations from one another, and which may be connected by a wireless connection (e.g., over a network, or internet connection). Each computing device 62a, 62b may have a processor 64a, 64b and a memory 66a, 66b. The processors 62a, 62b may be configured to perform method 50 (of FIG. 2), e.g., based on instructions stored in memory 66a, 66b.

The process requirements and downtime constraints received at the computing system 60 may be received from an external device (not shown in FIG. 3), e.g., via an input device 68, which may be a wireless received for example. The external device may be another computing device, such as a server or external memory device, for example.

The input device 68 may comprise a user interface, such as a keyboard, touchscreen, microphone etc., for receiving the process requirements and downtime constraints by user/operator input.

After receiving the process requirements and downtime constraints from an external source, they may be stored in one or more of memory 66a, 66b.

Alternatively, the process requirements and downtime constraints may be received from within the computer system. For example, a processor 64a, 64b of the computer system 60 may receive the process requirements and downtime constraints from memory 66a, 66b of the computer system 60.

Returning to method 50 of FIG. 2, at S506, a shortest sequence of steps of the drug production process is identified by the computing system 60, based on the process requirements and the downtime constraints. This comprises identifying the total duration of the sequence of steps for a plurality of different candidates of the sequence of steps, and selecting the sequence of steps with the shortest total duration as the shortest sequence of steps (see e.g., S507). The different candidates may comprise the sequence of steps performed at different times, or in different orders for example.

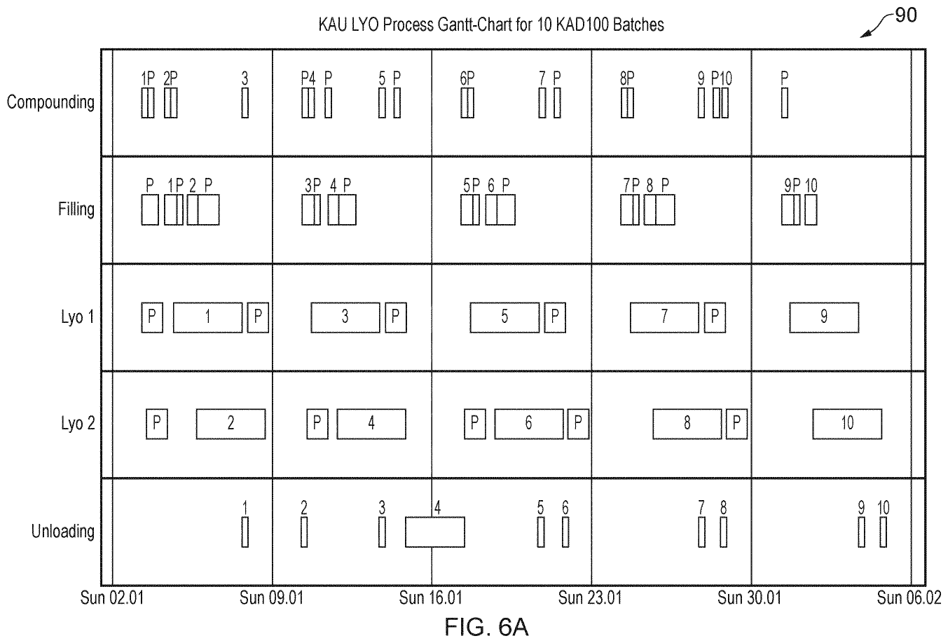

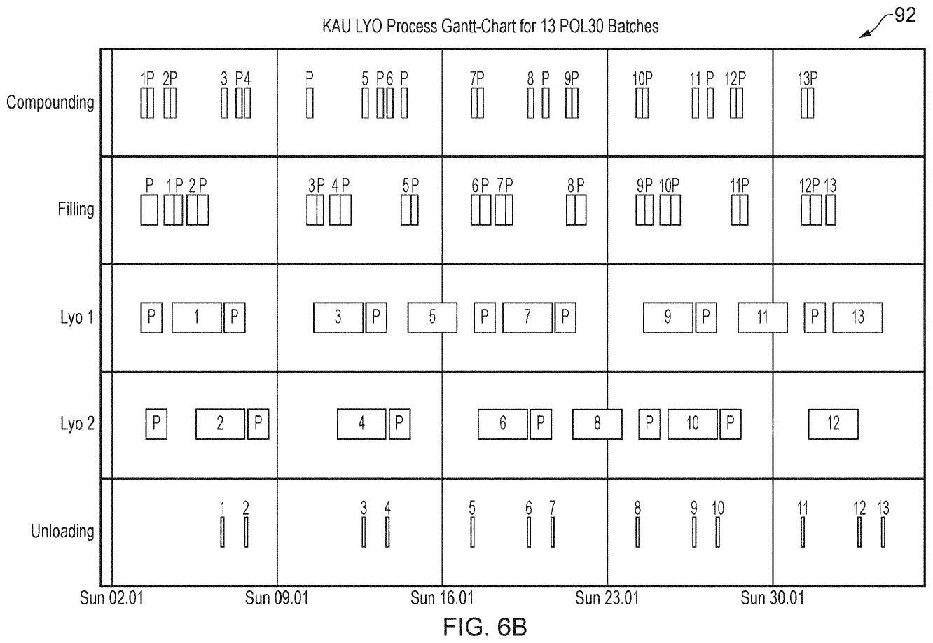

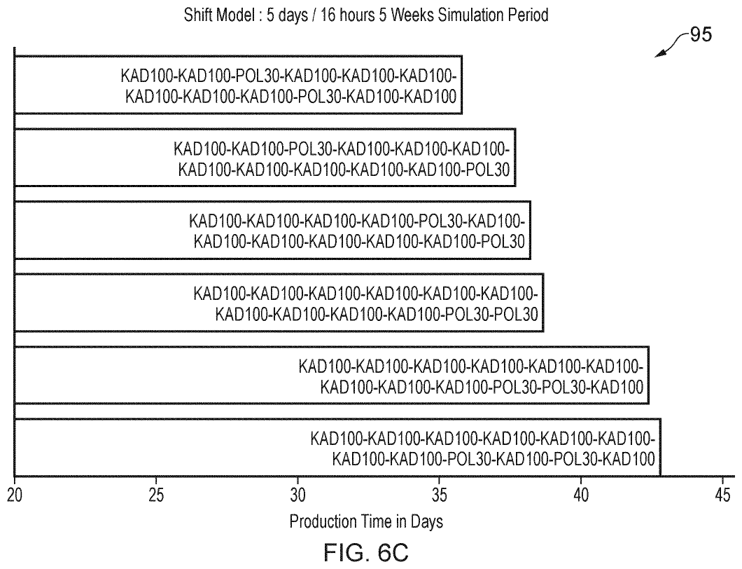

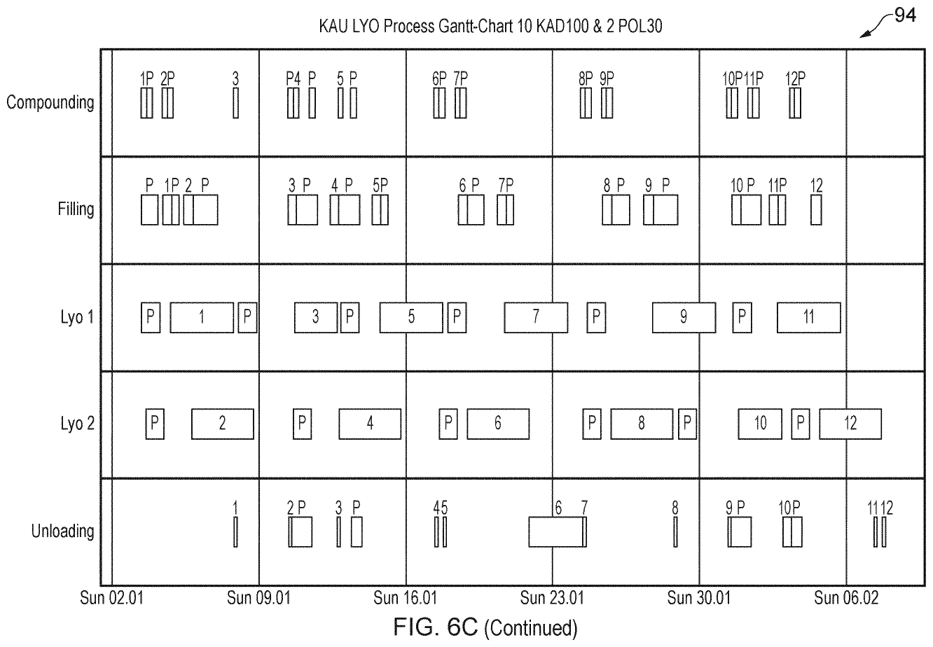

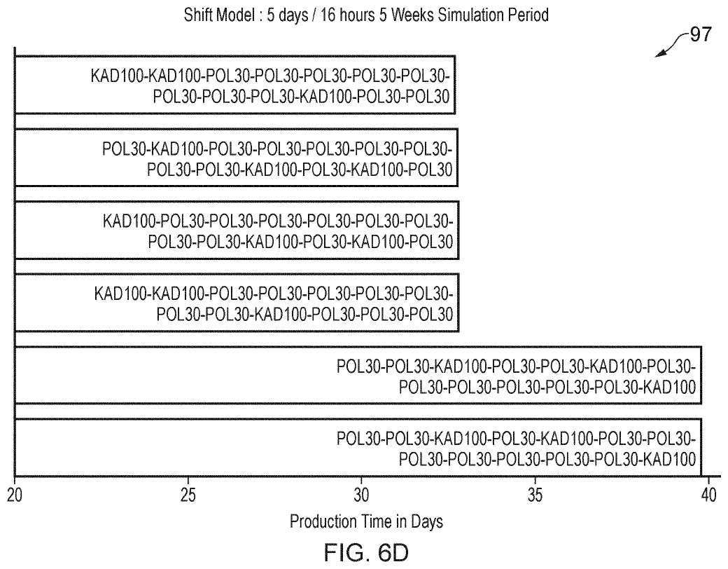

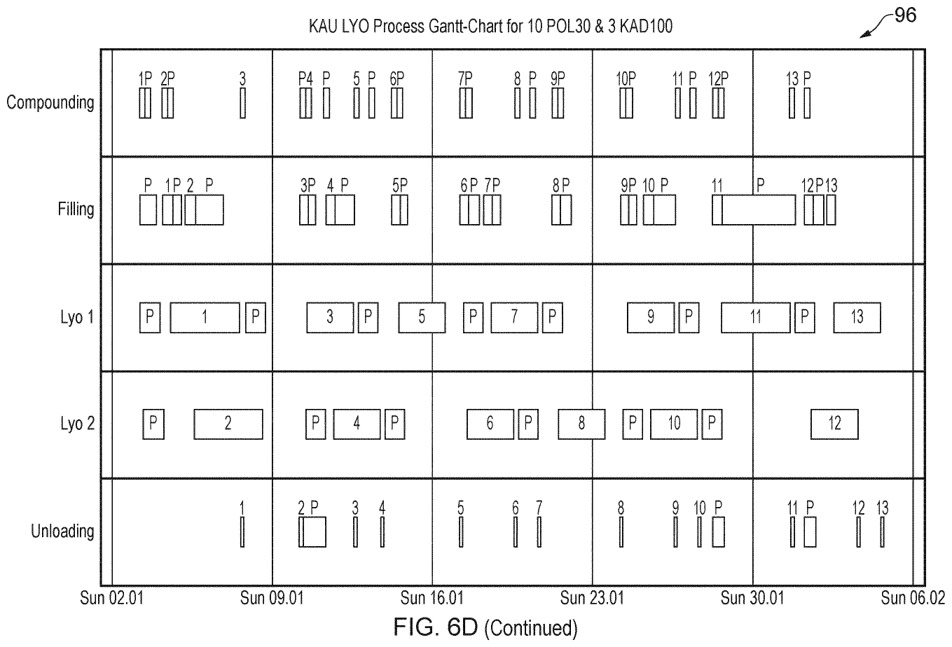

Although not shown in FIG. 2, the method may comprise outputting the identified shortest sequence of the drug production process, e.g., for display. For example, a display screen of the computing system 60 may display the identified shortest sequence of the drug production process. It may be displayed as a Gantt chart, such as the Gantt charts shown in FIGS. 6B-6D, which are described in further detail later.

Returning to S506, in some examples, identifying the shortest sequence of steps may comprise identifying a start time which provides the implementation of the drug production process with shortest total duration (e.g. the quickest implementation of the drug production process).

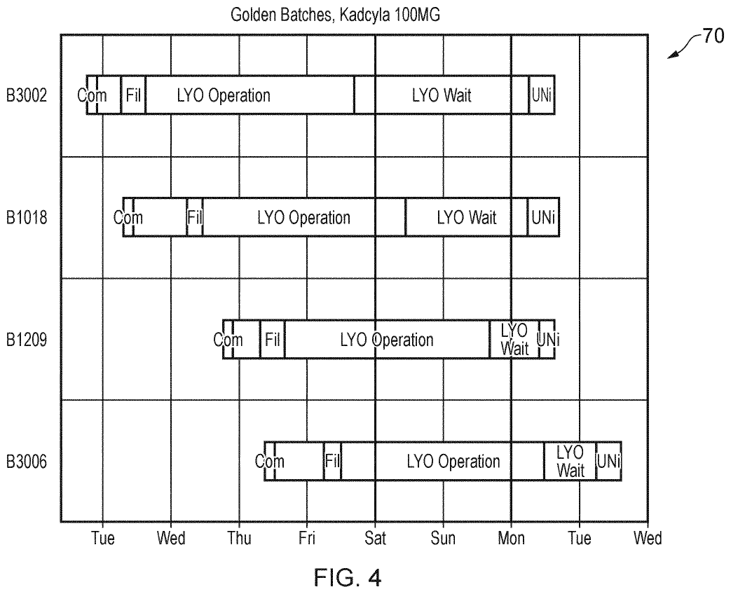

The Gantt chart 70 shown in FIG. 4 illustrates how the start time of the drug production process influences the total duration of the drug production process. In this example, the downtime constraints may be that the operators may be unavailable during the evenings and weekends. In these periods, certain portions of steps may be able to be implemented, but others may not. For example, the lyophilization step 30 may not be able to be initiated during these time periods, but the remainder of the lyophilization step 30 may be able to be performed in the operator's absence. This is shown in the drug production process marked B3006 in FIG. 4. Here, the lyophilization process 30 began before the period when the operator was unavailable (Saturday-Sunday), but was able to continue during that period despite the operator unavailable. The operator may be required for the initialisation of the unloading step 40. Thus, when the lyophilization process 30 ends during the period when the operators are unavailable, there is a complete downtime period where no steps are performed and so during this period the drug production process does not progress. This is illustrated in the drug production processes of FIG. 4 labelled B3002, B1018 and B1209. As such, it is shown that identifying the start time which provides the performance/implementation of the drug production process with the shortest total duration, and then starting the drug production process at that start time, would increase batch capacity of the drug product over a same period of time.

As mentioned above, the drug production process may produce two or more drug product using a respective lyophilization step. As such, the process requirements received in S502 may include the required steps of the drug production process for each of the at least two drug products. The different drug products may have different formats. The steps in the drug production process for the different drug products may have different approximate durations.

The process requirements may also include sequence requirements, for example a requirement that one of the drug products must be produced within a predefined time period.

Where the drug production process produces two (or more) drug products, identifying a shortest sequence of steps (in S506) may comprise identifying an order for producing the two drug products which results in the shortest sequence of steps, based on the process requirements and the downtime constraints. Identifying the order which results in the shortest sequence of steps may comprise identifying the total duration of the sequence of steps of the drug production process for a plurality of different candidates of the order of steps, and selecting the sequence of steps with the shortest total duration as the order which results in the shortest sequence of steps (e.g., as the quickest order).

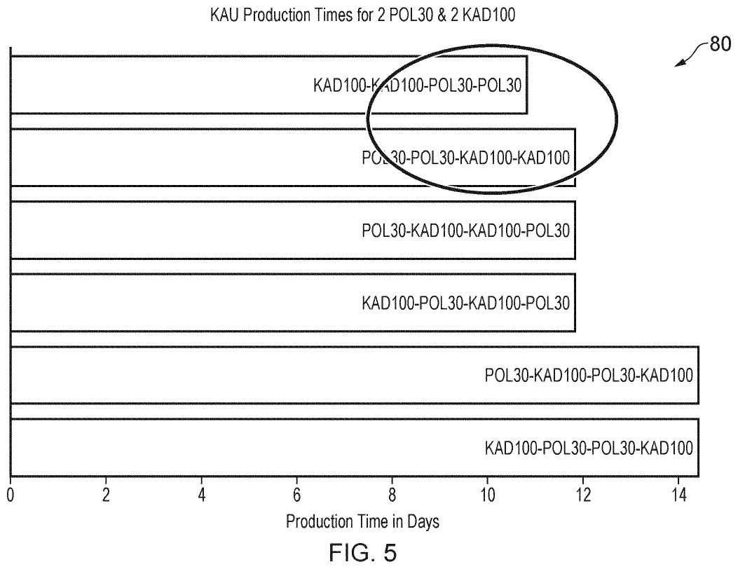

This is illustrated in bar chart 80 shown in FIG. 5.

In FIG. 5, the drug production process is for producing 2 batches of Polivy™ (an anti-CD79b antibody-drug conjugate (ADC) for patients with relapsed or refractory diffuse large B-cell lymphoma), with a format of 30 mg; and 2 batches of Kadcyla® (an antibody-drug conjugate engineered to deliver potent chemotherapy directly to HER2-positive cancel cells), with a format of 100 mg. Hereinafter, Polivy™ with a format of 30 mg is referred to as POL30, and Kadcyla® with a format of 100 mg is referred to as KAD100.

For each possible order, the total duration of the sequence of steps may be calculated based on the process requirements and the downtime constraints. In the example shown in FIG. 5, sequence KAD100-KAD100-POL30-POL30 is calculated to have a total duration of approximately 11.5 days, whereas sequence KAD100-POL30-POL30-KAD100 is calculated to have a total duration of approximately 14.5 days. As such, the quickest order is found to be KAD100-KAD100-POL30-POL30, which has a shorter duration than POL30-POL30-KAD100-KAD100 by approximately 24 hours (without any change in downtime constraints or amount of drug production produced). By decreasing the time required to produce the same quantity of drug product, batch capacity can be increased.

The advantages of the methods disclosed herein can be further described with reference to FIGS. 6A-6D.

FIG. 6A is a Gantt chart 90 showing an example previous (e.g. prior art) sequence of steps of a drug production process over a 5-week period. This sequence of steps allows 10 KAD100 batches to be produced over the 5-week period.

In contrast, FIG. 6B is a Gantt chart 92 showing a sequence of steps performed according to the methods disclosed herein. In this example, predefined start and end times were included in the process requirements (corresponding to the same start and end times in FIG. 6A). In this example, S506 of identifying the shortest sequence of steps of the drug production process comprises identifying a maximum batch capacity of the drug product based on the process requirements and the downtime constraints (including the predefined start and end time). By optimizing the sequence of steps (by identifying the shortest sequence of steps), the methods disclosed herein allow for 13 POL30 batches to be produced over the same 5-week period. Previously, only 10 batches would be produced.

FIG. 6C includes a bar chart 95 illustrating how the above described methods allow for 2 additional POL30 batches to be produced in the same time period as that in the example prior art sequence of steps shown in FIG. 6A. In particular, by identifying the shortest sequence of steps (and thus reducing the total time period for performing the drug production process), further drug products can be produced in the same period. Bar chart 95 shows how the quickest order is determined (as described above with reference to FIG. 5). Gantt chart 94 of FIG. 6C shows this quickest order over the 5-week time period.

FIG. 6D includes a bar chart 97 illustrating how the above described methods allow for 3 POL30 batches to be replaced with 3KAD100 batches without any time loss compared to the example described above with reference to FIG. 6B. In particular, the above described methods may allow for the production of one or more batches or drug products to be replaced with another drug product, within a same time period. Bar chart 97 shows how the quickest order of this replacement drug production process is determined (as described above with reference to FIG. 5). Gantt chart 96 of FIG. 6D shows this quickest order over the 5-week time period.

Returning to method 50 of FIG. 2, the method further comprises implementing the drug production process according to the shortest sequence of steps in order to produce the at least one drug product (S508). Accordingly, the amount of time required to product the drug product(s) is reduced, thus allowing for increased batch capacity over a same period, without modifying the downtime constraints, and whilst still obtaining the preferred quality resulting from the inclusion of the lyophilization step 30.

Although not shown in FIG. 2, the method may further comprise further steps prior to the step S508 of implementing the drug production process. In particular, the method may comprise identifying, at the computing system, a predicted total duration of the drug production process based on the identified shortest sequence of steps and historical data indicative of an actual duration of each step (or the sequence of steps) when implemented previously in the production of the same drug product(s).

The predicted total duration of the drug production process may be output, e.g., for display (in a similar manner as described above for the output of the identified shortest sequence of steps).

The method may comprise comparing, by the computing system 50, the predicted total duration of the drug production process to a predefined duration threshold. Optionally, the drug production process may only be implemented according to the identified shortest sequence of steps when the predicted total duration of the drug production process is less than the predefined duration threshold. In this way, the drug production process may only be implemented when the predicted total duration time is less than a predefined limit (e.g., when it is predicted that the process will finish within an operator's availability). This may reduce the need for operators to extend their working hours, for example.

Optionally, the predicted total duration of the drug production process may be identified (e.g., by the computing system) a plurality of times, using different historical data indicative of an actual duration of each step when performed previously in the production of the same drug product(s). The actual durations of a respective step implemented previously (e.g. in multiple drug production processes for the same drug product) may differ, e.g., based on external factors such as efficiency of the operators, or environmental conditions. Thus, when the predicted total duration is identified multiple times, the predicted total duration may differ based on this differing historical data. Then, the method may comprise determining, at the computing system, an average (e.g. mean, median) of the plurality of predicted total times and comparing the average to a predefined duration threshold, as detailed above.

Alternatively, the method may comprise comparing each of the plurality of predicted total times to a predefined duration threshold, and identifying an indicator representing the percentage of times the predicted total time is less than the predefined duration threshold. This indicator may indicate the probability of implementing the drug production process according to the identified shortest sequence of steps within a predefined time duration, and may be useful in risk analysis, e.g., in indicating the probability of the process overrunning or finishing within a preferred timeframe. The indicator may be output, e.g., for display (in a similar manner as described above for the output of the identified shortest sequence of steps).

Optionally, the historical data used in the predicted total duration of the drug production process may be limited to historical data of the duration of the steps implemented under the same downtime constraints as those received at the computing system. Advantageously, this allows for the simulation to better map the relationship between the durations of the steps of the drug production process for that specific drug product and the downtime constraints.

The method may comprise implementing the drug production process according to the identified shortest sequence of steps only when the indicator is greater than a predefined indicator threshold. In this way, the drug production process may only be implemented when there is a high chance of the drug production process being implemented within a predefined time period.

In further examples, the method may comprise identifying possible maintenance/cleaning periods for one or more pieces of equipment used in the drug production process, based on the shortest sequence of steps. In particular, the shortest sequence of steps may be analysed to identify any unavoidable downtimes of the equipment and may identify these periods as possible maintenance/cleaning periods. The possible maintenance/cleaning periods may output, e.g., for display (as described above).

A further implementation of the method is described below.

According to this implementation, the process requirements received (e.g., as input to the simulation) may comprise a time period of interest, a shift model (e.g., Monday-Friday 6 am-10 pm), potential non-production days (e.g., maintenance days, holidays), production order constraints (e.g., first batch needs to be product B) and/or product information.

For the product information, a name of the product, a number of batches and a number of plates that go into the lyophilizer may be required. An example of this product information is shown in Table 1 below.

TABLE 1

| Product | Batches | Plates |
|---|---|---|
| A | 2 | 10 |
| A | 1 | 7 |
| B | 4 | 14 |

In the above example in Table 1, there are 2 batches of product A with 10 plates, 1 batch of product A with 7 plates and 4 batches of product B with 14 plates to be simulated.

The simulation may treat batches listed in the same row as interchangeable, e.g., the order of the two product A batches with 10 plates is not fixed (and does not matter). However, the order of all three A batches is important (and thus does matter) as batches that require a different number of plates may require different process times (e.g., filling 10 plates requires more time and resources than filling 7 plates). Given the above product table and the production order constraints, the above-described simulation seeks to find the best product permutation, e.g.:
1. A(10)-A(10)-A(7)-B(14)-B(14)-B(14)-B(14)
2. A(10)-A(7)-A(10)-B(14)-B(14)-B(14)-B(14)
3. A(7)-A(10)-A(10)-B(14)-B(14)-B(14)-B(14)
4. And so on . . .

Resolving a simulation for a given list of batches may be a deterministic process that allocates the resources for the tasks in a given order at earliest possible times while respecting (e.g., bound by) any constraints defined for the process. Optimization may be built on top of scheduling and aims to find the optimal initial settings that results in a most desirable outcome.

For a given permutation and a given calendar (e.g., known working hours, holiday, maintenance days, available resources, etc.) the algorithm configured to perform the above-described simulation method may fill a given process at the earliest possible time slot. The earliest possible time slow for a process may depend on multiple factors. For example, it may depend on the completion of a previous process, it may require enough time during the working day to be completed, and/or it may require the presence of an employee with a specific skill, etc. However, it may be that a process must be scheduled in a way that it complies with product specific holding times (e.g., the filling preparation must have been completed within the last 7 days before filling, otherwise the filling preparation needs to be repeated). Given these constraints and dependencies, as well as the underlying calendar, the simulation may provide a schedule for a given permutation (e.g., a given candidate) by placing each process in a way that will finally provide the optimal schedule. Based on this schedule, the end-to-end lead time as well as additional statistics of interest, such as the overall equipment effectiveness or the asset utilization, may be calculated. The simulation may provide these results for each tested permutation (e.g., candidate) together with the corresponding schedule and can provide the user the opportunity to select/choose the production plan that best fits the current situation/their requirements.

The various constraints and special cases of the different production lines make it likely that the optimization problem does not fall neatly into any existing category of problems that would have a known good solution in the literature. Use of an evolutionary algorithm is preferred to address this issue. The evolutionary algorithm may comprise custom mutation and reproduction steps that have been shown to find good solutions with a manageable computation time.

The most common optimization problem in a scheduling context is finding the permutation of product batches that leads to the shortest lead time. This is a combinatorial problem that can usually be solved reasonably well with brute force or some variation of a tree search algorithm if the number of batches and batch types is not too large and if the constraints are not too complex. In the general case, however, the number of solutions is prohibitively large for any type of exhaustive search. Accordingly, use of an evolutionary algorithm that relies on randomness and heuristics for exploring the solution space and has shown to provide us with good solutions in a reasonable time, is preferred.

Evolutionary algorithms are inspired by natural selection and borrow terminology and ideas from that field. There is a plurality of variations of evolutionary algorithms. One such preferred algorithm for the above-described method may be used as follows. The algorithm may start with a randomly sampled “population” of solutions, e.g., 200 random permutations of the batches. The “fitness” of these solutions is evaluated by feeding them to the scheduler and measuring the simulated lead time. The top performing “elite” solutions survive to the next iteration and get to “reproduce” which in practice means picking two random parents from the elites and combining their features in some manner to produce new solutions. In permutation problems, this may not be straightforward. A partially-mapped crossover (PMX) may be used to address these issues. Offspring can also incur random “mutations” that add random noise to the solutions. There are multiple options but in the scheduling context it may be preferred to try solutions where the places of two batches are swapped, a chunk of batches from the tail of the sequence are moved to the front or a chunk of batches is moved to a random position. The rationale behind these mutations is to keep much of the order intact and to try exploring solutions that are in scheduling sense close to the original. Once enough offspring have been generated to replenish the population to its original size the process is repeated starting by evaluating the fitness of the new population. This cycle is continued until the best observed fitness has stalled for a given number of iterations or a predefined maximum number of iterations is reached.

For a typical long-term simulation use case that schedules 50-100 batches over a span of a year, a population of 200 solutions may be used and 200 generations may be run to get improvements of 10%-20% compared to the average random permutation. The main bottleneck of the algorithm, that is the evaluation of the fitness function, can be run in parallel.

In addition to outputting (e.g., for display) an end-to-end lead time of candidate, the above-described simulation may also output additional information that may be useful for a user in choosing a most appropriate production plan. The additional information may include, for each of a plurality of candidates, the number of product changeovers (e.g., changing from product A to product B), the overall equipment effectiveness, the asset utilization, and the number of batches with high risk of overtime hours, for example. The additional information may also indicate the candidate with the minimum change over time (e.g., changing from product A to product B). This may indicate a candidate with a higher probability to succeed.

The process requirements may additionally include information about operators including specialised skills associated with the operators. For example, certain processes can only be started if a predefined number of operators are available (e.g., filling may require four people), and some processes may also require the availability of an operator with a special skill (e.g., filling may require 3 operators and 1 technician). Including this information about the operators in the process requirements received at the computer system and used in the simulation, can improve the accuracy of the output.

Optionally, different steps in the drug production process may have different schedule constraints. For example, compounding may be possible for 24 hours a day, 7 days a week, whereas filling may only be possible 24 hour a day, 5 days a week. These schedule constraints may be included in the received downtime constraints.

Additional constraints may be received and used in the identification of the total duration of the drug production process for a plurality of different candidates for a sequence of steps of the drug production process. For example, an additional constraint to be met may be a predefined maximum number of batches of the same product. Additional constraints may also include a predefined distribution of batches for a given time period (e.g., in a given drug production process, a predefined ratio of products A and B should be produced).

The above-described methods can account for variability within a process, and thus can account for the total variability of an entire production plan. Determining the variability of a drug production process can provide crucial insight about the feasibility of a drug production process. This information can be crucial since a plan might be feasible in theory, but in the end unlikely to successfully implement in reality. In addition, the entire model can be run with x % prolonged process durations to provide some additional tolerance that might be required due to unplanned events.

The above described methods may also be applied to portions of drug production processes (e.g., only a portion of the drug production 10 described above with reference to FIG. 1). Accordingly, if the drug production process has been halted at some point during the process due to an unplanned event, the methods disclosed herein can be repeated for the remaining steps, thus allowing for the production line to be resumed more quickly, identifying the shortest sequence of events for the remaining steps of the process. For example. If the filling preparation took 5 hours longer than expects, and as a result, the filling needs to be pushed back a few hours or even to the next day, the simulation can provide the new optimal sequence of steps given this additional constraint.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A method for producing at least one drug product using a lyophilization step, the method comprising:
receiving, at a computing system, process requirements of a drug production process for the at least one drug product to be produced, the drug production process comprising at least one pre-processing step, the lyophilization step, and at least one post-processing step, wherein the process requirements comprise:
the required steps of the drug production process for the/each drug product;
operator requirements of each step; and
an approximate duration of each step;
receiving, at the computing system, downtime constraints of the equipment required for the drug production process, wherein the downtime constraints comprise time periods when the lyophilization step cannot be initiated, but where at least one or more steps, or portions of steps, of the drug production process can be implemented;
identifying, at the computing system, a total duration of the drug production process for a plurality of different candidates for a sequence of steps of the drug production process, based on the process requirements and the downtime constraints;
identifying, at the computing system, a shortest sequence of steps of the drug production process by selecting, from the plurality of different candidates, the sequence of steps with the shortest total duration as the shortest sequence of steps; and
implementing the drug production process according to the shortest sequence of steps in order to produce the at least one drug product.

2. The method of claim 1, wherein the at least one pre-processing step comprises a compounding step and/or a filling step; and the at least one post-processing step comprises an unloading step.

3. The method of claim 1, further comprising determining, at the computing system, the approximate duration of each step by:
receiving, for each step, historical data indicative of an actual duration of the respective step when implemented previously;
determining, for each step, an average duration of the historical data; and
determining the approximate duration of each step based on the respective average duration.

4. The method of claim 3, wherein the historical data is limited to historical data of the drug production process implemented under the same downtime constraints as those received at the computing system.

5. The method of claim 1, wherein the downtime constraints include time periods when the equipment required for one or more steps, or portions of steps, is unavailable.

6. The method of claim 1, wherein identifying the shortest sequence of steps comprises identifying a start time which provides the implementation of the drug production process with the shortest total duration.

7. The method of claim 1, wherein the process requirements comprise a predefined start time and a predefined end time of the drug production process.

8. The method of claim 7, wherein identifying the shortest sequence of steps of the drug production process comprises identifying a maximum batch capacity of the drug product based on the process requirements and the downtime constraints

9. The method of claim 1, wherein the method is for producing at least two different drug products, the required steps of the drug production process for each drug product comprising a lyophilization step.

10. The method of claim 9, wherein identifying a shortest sequence of the steps of the drug production process for the at least two drug products comprises identifying an order for producing the at least two drug products which results in the shortest sequence of steps, based on the process requirements and the downtime constraints.

11. The method of claim 10, wherein identifying the order which results in the shortest sequence of steps comprises:
identifying the total duration of the sequence of steps of the drug production process for the at least two drug products for a plurality of different candidates of the order of steps; and
selecting the sequence of steps with the shortest total duration as the order which results in the shortest sequence of steps.

12. The method of claim 1, further comprising, before implementing the drug production process according to the identified shortest sequence of steps:
identifying, at the computing system, a predicted total duration of the drug production process based on the identified shortest sequence of steps and historical data indicative of an actual duration of each step when implemented previously in the production of the same drug product.

13. The method of claim 12, further comprising:
identifying the predicted total duration of the drug production process a plurality of times, using different historical data indicative of an actual duration of each step when implemented previously in the production of the same drug product;
comparing each of the plurality of predicted total times to a predefined duration threshold; and
identifying an indicator representing the percentage of times the predicted total time is less than the predefined threshold.

14. A computer system comprising one or more processors, the one or more processors configured to perform the method of claim 1.
